(19) United States
(12) Patent Application Publication
(10) Pub. No.: US 2006/0141472 A1
(43) Pub. Date: Jun. 29, 2006

(54) Medical use of ras antagonists for the treatment of capillary malformation

(76) Inventors: Miikka Vikkula, Kraainem (BE); Laurence Boon, Kraainem (BE); Liro Eerola, Brussels (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/546,692

(22) PCT Filed: Mar. 20, 2003
(86) PCT No.: PCT/EP03/02913

§ 371 Date: Sep. 28, 2005

(51) Int. Cl. C12Q 1/68

(52) U.S. Cl. 435/6

(57) ABSTRACT

The invention relates to the field of vascular anomalies and methods for diagnosing and treating them. The invention provides for the causative gene (RASA1) and mutations therein which are useful for diagnosis of inherited capillary malformations. The invention further provides RASA1 antagonists for use in treatment of capillary malformations.

### FIELD OF THE INVENTION

The invention relates to the field of vascular anomalies and methods for diagnosing and treating them.

### BACKGROUND OF THE INVENTION

Defects in cutaneous vascular development are manifested as vascular anomalies or malformations that vary in size, anatomic location, internal blood flow and clinical severity varying from life-threatening lesions to cosmetic harm. They are localized defects of vasculogenesis and/or angiogenesis. Capillary malformation (CM) in the form of “port-wine stain” is the most common vascular malformation occurring in 0.3% of newborns. CMs are small flat cutaneous lesions that consist of capillary-like channels that are dilated and/or increased in number in the dermis (Barsky et al., 1980). Vascular birthmarks, such as salmon patch, are milder variants of CM that occur in up to 40% of newborns. Unlike common macular stains, the reddish coloration of CMs does not disappear, but becomes darker with advancing age. Arteriovenous malformation (AVM) and arteriovenous fistula (AVF) are fast-flow vascular anomalies that affect the skin, other soft tissues, bones, internal organs and brain, and can cause life-threatening complications, such as congestive heart failure, severe bleeding or neurologic consequences. Multiple AVFs co-occur with cutaneous CM and soft tissue and skeletal hypertrophy of the affected limb in Parkes Weber Syndrome (Mulliken et al., 1988).

Increased incidence of lesions in first-degree relatives of CM patients and several reported familial cases revealed an autosomal dominant inheritance and suggested that genetic actors may play a role in the pathogenesis of CM (Eerola et al., 2001).

It is an aim of the present invention to provide new medicaments and new therapies for reating vascular anomalies.

### DESCRIPTION OF THE INVENTION

The present inventors performed a genome wide linkage analysis performed on families with inherited CM. In non-parametric linkage analysis, statistically significant evidence of linkage (peak Z-score 6.72, p-value 0.000136) was obtained in an interval of 69 cM on 5q11-5q23. Parametric linkage analysis gave a maximum combined HLOD score of 4.84 (α-value 0.67) from the same region and the analysis using only the linked families, defined a smaller, statistically significant locus CMC1 of 23 cM (LOD score 7.22) between markers D5S1962 and D5S652 corresponding to 5q13-5q15 (Eerola et al., 2002: the complete document is included herein by reference). Interesting candidate genes implicated in vascular and neuronal development, such as MEF2C (mycocyte enhancer factor-2C), RASA1 (RAS p21 protein activator-1), and THBS4 (thrombospondin-4 gene) are in this locus. Mice deficient in MEF2C manifested lumen size abnormalities of the large vessels close to the heart, as well as diminished peripheral capillary vasculature (Bi et al., 1999). Mosaic murine embryos composed of wild-type and RASA1 null cells exhibited localized vascular defects (Henkemeyer et al., 1995).

A new family (family CM45, FIGS. 1 and 2) made it possible to narrow the linked locus to 5 cM, between markers D5S459 and GATA5F09. This interval contains eight characterized genes, three of which, RASA1, EDIL3 (EGF-like repeats and discoidin I-like domains 3) and MEF2C, are now considered to be functionally interesting candidate genes.

It is herein described how positional candidate gene analysis in this large region led to the identification of the mutated gene that seems to cause pathological angiogenesis by haploinsufficiency. Mutations in RASA1, the gene encoding p120-RasGAP, can cause CMs, AVMs, AVFs and Parkes Weber Syndrome. Specific mutations are described further.

The p120 Ras GTPase-activating protein (p120-RasGAP) is a modular protein of 1047 amino acids containing two SH2 and an SH3 domain in the N-terminal region, a pleckstrin homology domain and a protein kinase conserved region 2 in the central region, and a RasGTPase activating domain in the C-terminal region (FIG. 3). Alternative names for the p120 Ras GTPase-activating protein are p120-RasGAP and RAS p21 activator 1 and are used interchangeable throughout the application.

P120-RasGAP is best known for its function as a negative regulator of Ras/MAPK signaling pathway that mediates signals for cellular growth, differentiation and proliferation from various receptor tyrosine kinases (RTK) on the cell surface (Hanahan and Weinberg, 2000) P120-RasGAP turns the active GTP-bound-Ras to the inactive GDP-bound form (Denhardt, D. T., 1996). The Ras protein is a guanine nucleotide binding protein. In response to growth factor stimulation of the cell, Ras proteins become bound to GTP instead of GDP, stimulated by the Ras specific exchange factors.

The disorder that is caused by mutations in RASA1 is an entity that as such has never been described in medical literature. The disorder is defined by the association which the inventors identified between the hereditary capillary malformations, arteriovenous-malformations (AVM), arteriovenous-fistulas (AVF) and Parkes Weber syndrome. This newly defined type of disorder will be generally referred to as CM-AVM.

As capillary malformations, in general, are frequent in the population (0.3%), RASA1 mutations can be more common than currently shown by this study where 4 families were identified in the relatively small population of the Walloon part of Belgium.

The results herein presented show that an atypical cutaneous capillary malformation, e.g. inherited capillary malformations, can be an indicator of a genetic susceptibility to more severe internal vascular malformations, namely, AVM, AVF and Parkes Weber syndrome.

AVMs have always been considered to be non-hereditary, the present inventors herein showed that AVM can be caused by genetic predisposition, namely by a mutation in the RASA1 gene.

Latent intracranial AVM and carotid AVF can cause life-threatening hemorrhage, malformations that are conventionally identified by screening at risk individuals by MRI (magnetic resonance imaging) or Echo-Doppler-examinations.

The expression “vascular anomalies” is to be understood as a very broad definition and comprises any defect (congenital or acquired) affecting the morphology, structure, location, or function of blood or lymphatic vessels. Vascular anomalies in the skin are often referred to as “birthmarks” (which is an imprecise lay term). Vascular anomalies in the skin (=cutaneous vascular anomalies) are divided into two groups: vascular tumors (hemangiomas) and vascular malformations. Vascular malformations are further divided on the basis of the clinical phenotype and the vessel type affected into capillary, venous, arterio-venous and lymphatic malformations (Mulliken and Glowacki, 1982; Mulliken and Young, 1988). Each of these subgroups may contain several variants, e.g. venous malformation group contains sporadic venous malformation, inherited cutaneomucosal venous malformation, glomuvenous malformation and blue rubber bleb nevus syndrome.

The abbreviation “CM” means capillary malformation, a commonly used synonym is port-wine stain. A cutaneous CM is a CM located in the skin.

As explained above, the term “birth marks” is also broadly used, for instance to indicate what is called a salmon patch, which is a cutaneous vascular stain that fades and disappears by time. Other synonyms are angel's kiss and nevus flammeus neonatorum.

The abbreviation “AVM” means arterio-venous malformation, which is a fast-flow vascular anomaly, which is constituted of a so called “nidus” which is fed by possibly several feeding arteries, and drained by some veins. The nidus creates a direct connection between the arterial and venous part of the vasculature without normal intervening capillaries.

The abbreviation “AVF” means arterio-venous fistula, which is a fast-flow vascular anomaly consisting of a direct vascular connection between an artery and a vein, without a nidus, contrary to AVM, and without normal capillary network in between. AVF thus constitutes another form of direct connection between an artery and a vein.

The expression “Parkes Weber Syndrome” relates to a syndrome which usually affects one extremity, but which can also be bi-lateral. The affected limb contains multiple small AVFs associated with hypertrophy (or hypotrophy) of the affected limb. Usually a CM can be observed on the skin.

The present inventors clearly identified a heritable association between cutaneous vascular anomalies and more severe internal anomalies by the identification of mutations in the RASA1 gene. Individuals showing atypical cutaneous capillary malformations not only should be referred to MRI or Echo-Doppler-examinations, but can now be screened for genetical predisposition for acquiring more severe internal anomalies.

The present invention thus provides new tools for diagnosing severe vascular malformations at the molecular level.

The human RASA1 gene is located on chromosome 5. For the moment, the complete sequence of the human genome is publicly available, for instance, for the region of chromosome 5 containing the RASA1 gene, under the accession number NT_{—}037660 and retrievable from http:/www.ncbi.nim.nih.gov/. The cDNA sequence of RASA1 is available under the accession number NM_{—}002890.1 and is depicted in FIG. 8 as SEQ ID NO 1. The corresponding amino acid sequence is depicted in FIG. 8 as SEQ ID NO 2. It should be clear that individual and allelic differences in nucleic acid and/or amino acid sequence are possible as well as possible gene duplications. Therefore, whenever reference is made to a genomic nucleic acid encoding RASA1, all normally occurring sequence variants should be covered which are able to encode the RAS p21 activator 1. The representation by SEQ ID NO 2 for the wild type RAS p21 activator 1 protein is only one occurring alternative existing in nature and representing a normal functioning RAS p21 activator 1.

According to a first embodiment, the invention relates to a method for diagnosing inherited capillary malformation using a nucleic acid substantially complementary to a nucleic acid sequence in the RASA1 gene, for instance the genomic sequence on chromosome 5. Preferably said nucleic acid has a sequence of at least 10 or at least 15 contiguous nucleotides chosen from the RASA1 genomic sequence, for instance a sequence that can be retrieved from the genbank and encoding RAS p21 protein activator 1, represented by SEQ ID NO 2, or the complement of said nucleic acid sequence. Preferably said nucleic acid is complementary to a region of the RASA1 gene wherein a mutation may occur, which mutation is inherited by persons showing vascular anomalies. Preferably said nucleic acid is a probe or primer. As the penetrance is not necessarily 100%, some people may carry a RASA1 mutation without having vascular anomalies.

Whenever herein reference is made to “the RASA1 gene” this relates to any nucleic acid sequence which for instance can be retrieved from the genbank and which encodes the natural RAS p21 protein activator, represented by SEQ ID NO 2. In the present application, the RASA1 gene may refer to its coding or to its non-coding strand, or may refer to both strands.

The invention further relates to a probe for in vitro diagnosing vascular anomalies in a subject carrying a mutation in the RASA1 gene, with said probe containing a sequence constituted of at least from about 10 successive nucleotides substantially complementary to a sequence in the RASA1 gene wherein one of the following deletions or mutations may occur: RASA1Δ^{CT593-594}, RASA1Δ^{GTCT1697-1700}, RASA1Δ^{GC2454-2455}, RASA1Δ^{T630}, RASA1^{1454(C>T)}, RASA1^{1737(G>A)}. The numbering of the nucleotide bases (in upper script) relates to the numbering as in the cDNA encoding RASA1, and represented in SEQ ID NO 1. Preferably allele-specific probes complementary to the mutated region can be used as hybridization probes. Preferably said probes are labeled with a detectable marker. The said probes can be used in a method for in vitro diagnosing vascular anomalies.

In a more specific embodiment the invention relates to a method for in vitro diagnosing vascular anomalies in a subject carrying a mutation in the RASA1 gene, said process comprising the step of detecting a mutation in the RASA1 gene in at least one of the positions RASA1Δ^{CT593-594}, RASA1Δ^{GTCT1697-1700}, RASA1Δ^{GC2454-2455}, RASA1Δ^{T630}, RASA1^{1454(C>T)} RASA1^{1737(G>A)} and by bringing into contact DNA, isolated from a biological sample taken from a patient, with a probe as described above, with said contact being carried out under conditions enabling the production of hybridization complexes formed between said probe and said DNA and detecting the above hybridization complexes which have possibly been formed.

The probes used in any of the methods herein described may by detectably labeled.

In mini-sequencing experiments, a sequencing primer is hybridized next to the mutation site, followed by a 1-nucleotide sequencing reaction. As both strands can be sequenced, the forward primer has a sequence corresponding to the coding sequence, the reverse primer has a sequence corresponding to the non-coding strand. Preferably the primers are about 20 nucleotides and hybridize to a sequence of about 20 nucleotides before or after the mutation site.

According to a further embodiment, the invention relates to a method for diagnosing inherited capillary malformation using at least one nucleic acid substantially complementary to a sequence in the RASA1 gene, for instance a sequence encoding the RAS p21 activating protein 1 as represented in SEQ ID NO 2, or the complement of said sequence, characterized in that said sequence in the RASA1 gene is flanking the region wherein a mutation may occur, said mutation being inherited by persons showing vascular anomalies. Preferably said sequence when used as a pair of sequences, i.e. primer pair will amplify the sequence of the RASA1 gene comprising the mutation.

The invention also relates to primers for use in the above method. More specific the invention relates to a primer for in vitro diagnosing vascular anomalies in a subject carrying a mutation in the RASA1 gene, with said primer containing a sequence constituted of from about 10 successive nucleotides specifically amplifying a region in the RASA1 gene wherein one of the following deletions or mutations may occur: RASA1Δ^{CT593-594}, RASA1Δ^{GTCT1697-1700}, RASA1Δ^{GC2454-2455}, RASA1Δ^{T630}, RASA1^{1454(C>T)}, RASA1Δ^{GIVS17+1} or RASA1^{1737(G>A)}.

Currently, each mutation can be identified by sequencing the amplicon of the corresponding mutated exon. The primers which can be used to amplify each of the 25 exons and in addition the isoform 2-specific exon 1, are represented in Table 1 by their respective SEQ ID NOs. For instance the primer pair RAS2F/RAS2R is used to amplify exon 2. Exon 1 is amplified in two separate fragments, and exons 16 and 17 are amplified in a single amplicon, using the primer pairs as explained in the Examples section.

For instance, the mutations RASA1Δ^{CT593-594} and RASA1Δ^{T630} are located in exon 1; mutation RASA1^{1454(C>T)} is located in exon 10; mutation RASA1Δ^{GTCT1697-1700} is in exon 11; mutation RASA1^{1737(G>A)} is in exon 12, mutation RASA1Δ^{GIVS17+1} is in the splice site of exon 17 and mutation RASA1Δ^{GC2454-2455} is in exon 17. Some of the mutations can be identified because they destroy the recognition sequence of certain restriction enzymes. For instance RASA1^{1454(C>T)} (Q446X), destroys a Sau3A1 restriction enzyme cutting site, and therefore can be detected by Sau3A1 digestion of the amplified fragments. Other mutations are recognized by different size of the amplified products on gel electrophoresis, and still other mutations are analyzed by allele-specific PCR, as shown in the figures and in the Examples section. The mutation RASA1^{1737(G>A)} (C540Y) in family CM11 does not change any restriction enzyme cutting site and is screened by allele specific PCR on both strands with primer pairs Ras12BF/Rmut & Ras12BF/Rwt, and Ras12R/Fmut & Ras12R/Fwt. When a mutation is present, the primer pairs Ras12BF/Rmut & Ras12R/Fmut will give a PCR product, otherwise only the wild-type primer pairs, Ras12BF/Rwt & Ras12R/Fwt will function.

The mutation RASA1Δ^{GIVS17+1} can be detected by sequencing the exon 16/17 amplicon.

The invention further relates to any of the above methods wherein the said vascular anomalies to be diagnosed are CM-AVM disorders, for instance a disorder selected from the group of atypical (here also called “inherited”) capillary malformation (CM), arteriovenous malformation (AVM), arteriovenous fistula (AVF) and Parkes Weber Syndrome. The invention also relates to the use of a nucleic acid of at least 10 contiguous basepairs chosen from the sequence of the RASA1 gene for in vitro diagnosing inherited vascular malformations. Specific primers are represented by SEQ ID NOs 3 to 61, as shown in Table 1.

The invention further relates to a kit for in vitro diagnosis of vascular anomalies in a subject carrying a mutation in the RASA1 gene, said kit comprising:
a determined amount of a nucleotide probe as defined above,
optionally primers to amplify a fragment of chromosome 5 comprising at least part of the RASA1 gene,
optionally, the appropriate medium for creating an hybridization reaction between the fragment and the probe,
optionally, reagents enabling the detection of hybridization complexes which have been formed between the fragment and the probe during any hybridization reaction.

The invention further relates to a kit for in vitro diagnosis of vascular anomalies in a subject carrying a mutation in the RASA1 gene, said kit comprising:
primers to amplify a fragment of chromosome 5 comprising at least part of the RASA1 gene,
optionally, the appropriate reagents for carrying out the amplification reaction,
optionally, a restriction enzyme for digestion of the amplification products,
optionally appropriate primers and reagents for sequencing the amplified fragment.

The kits of the invention may contain primers for diagnosing a specific mutations, and will therefore contain the specific primers for amplifying the exon where the mutation is to be suspected. Other kits may contain primers for amplification of more than one exon, preferably the exons where mutations are expected. Optionally said kits contain a restriction enzyme which is specifically used to detect a restriction length polymorphism in one or more of the amplified exons. A list of primers which are preferentially to be included in the kits of the invention is represented in Table 1.

The invention thus relates to any of said kits which can be used for the detection of genetic deletions or mutations associated with at least one condition selected from the group of CM-AVM disorders, for instance CM, AVM, AVF and Parkes Weber Syndrome, said kits comprising at least one probe and/or at least one primer as described herein.

The invention also relates to the use of a probe or a primer for the in vitro detection of a mutation in the RASA1 gene.

The invention also relates to methods for treating, preventing or alleviating vascular anomalies by restoring or replacing the lost protein function, more particularly restoring or replacing RASA1 activity via increasing the amount of the normal protein in the cell. The invention thus relates to a method of gene-therapy for treating, preventing or alleviating vascular anomalies using a nucleic acid encoding the RASA1 gene. Also envisaged are methods for treating, preventing or alleviating vascular anomalies by introduction into cells, in the bloodstream or in the body, an effective amount of the RASA1 protein to restore the normal level of inactive GDP-bound Ras protein in the cells, bloodstream or body.

Other useful methods according to the invention include methods comprising increasing the expression of the RASA1 gene in the non-defective, e.g. non-mutated allele, thus restoring a normal level of RASA1 protein in the defective cells or in the defective vascular tissues.

On the other hand, Ras antagonists or activity modifiers could have therapeutic potential in the disturbed downregulation of Ras signaling pathways in these patients. RASA1 protein is known as the activator of Ras GTPase, which means that if RASA1 mutations cause loss-of-function, the activity of RAS GTPase should be lower than normal, and thus Ras should stay in GTP-bound form longer, i.e. more active. Thus, the Ras overactivity is the first and most likely pathogenic alteration in individuals carrying vascular anomalies.

In addition other RASA1 mutations may cause qualitative alterations in the pathway of converting active Ras-GTP to inactive Ras-GDP.

The present invention provides medicaments comprising active substances for treating, preventing or alleviating vascular anomalies. These active substances act by restoring or normalizing the downstream effects of the lack of RASA1 protein, for instance downregulation of increased activity of Ras, or increasing the too low activity of Ras GTPase.

According to one embodiment, the invention relates to the use of a substance able to convert active GTP bound Ras protein into inactive GDP bound Ras protein intracellular in a cell for treating, preventing or alleviating vascular anomalies, or to the said use for the preparation of a medicament for treating, preventing or alleviating vascular anomalies.

The term “substance” as used herein relates to a compound, a mixture of compounds, a composition or the like.

According to the invention, said substance is a Ras antagonist or Ras activity modulator. Ras antagonists or Ras activity modulators could have therapeutic potential in normalizing the disturbed Ras signaling pathways in these patients.

The “Ras antoganists” of the present invention comprise all substances which weaken the signaling downstream of Ras, i.e. inhibit the signaling e.g. from tyrosine kinase (growth factor) receptors, via Ras towards the downstream intracellular effectors. Ras anatagonists may act in several ways, for instance a Ras antagonist could inactivate Ras, for instance by destroying or by modulating the Ras protein structure. A Ras antagonist may also inhibit Ras, for instance by binding to it or by slightly modifying its structure.

The “Ras activity modulators” not only comprise all substances which have a quantitative effect on Ras activity, for instance up- or downregulation of Ras, but also comprise substances which qualitatively modulate the Ras protein. We cannot exclude that some qualitative changes might also occur due to the RASA1 mutations. Such changes should primarily alter RASA1 function and only secondarily, e.g. via binding of the mutant RASA1, other molecules. The results of RASA1 mutations may be that Ras has altered activity towards certain downstream effectors, e.g. that it phosphorylates proteins that it normally does not phosphorylate or that it binds to proteins to which it normally does not bind to. Such altered interactions would change the downstream signaling specificity rather than activity.

In an alternative embodiment the invention relates to the use of a substance that converts active GTP bound Ras protein into inactive GDP bound Ras protein in a cell for treating, preventing or alleviating vascular anomalies.

The invention further relates to the use of a substance for treating, preventing or alleviating vascular anomalies or for the preparation of a medicament for said use, characterized in that said substance modulates the status of p120 RasGAP in a cell resulting in the presence of p120 Ras GAP protein in said cell in an amount effective to inactivate GTP bound Ras protein. According to a preferred embodiment said substance is a Ras antagonist or a Ras activity modulator.

In a further embodiment, said substance is chosen from the group of compounds that are:
1) Ras inhibitors, such as ISIS2503, farnesyl transferase inhibitors R115777, SCH66336 and BMS 214662;
2) compounds inhibiting the downstream effector Raf, such as ISIS 5132 or BAY 43-9006; or 3) compounds inhibiting MEK, such as C1-1040 (also known as: PD184352).

These compounds may act on Ras itself or act on the downstream effectores Raf and MEK. Several of these compounds are currently used in clinical trials for other Ras-related disorders (tumors) (for review: Dancey J E, Curr. Pharm. Des. 2002; 8 (25): 2259-67: Agents targeting ras signaling pathway; and Adjei A A. Curr. Pharm. Des. 2001 November; 7(16): 1581-94: Ras signaling pathway proteins as therapeutic targets; and Herrera r. and Sebolt-Leopold J. S. Trends in Molecular Medicine vol 8, no 4, (suppl.) 2002. S27-31: Unraveling the complexities of the Raf/MAP kinase pathway for pharmacological intervention). It should be clear that the present invention extends to all compounds described in these articles and which are shown to have an effect on the Ras signaling pathway proteins.

Although these Ras antagonists or Ras activity modulators, which are active downstream the Ras pathway may have been used in the field of treating Ras-related disorders, until now, they have not been used to treat vascular anomalies, such as the inherited capillary malformations described in the present invention. More specific, they have not been used as a therapeutic for treating diseases caused by mutations in the RASA1 gene.

The invention further relates to any of the above uses wherein said vascular anomalies are selected from the group of CM-AVM disorders, for instance capillary malformation (CM), arteriovenous malformation (AVM), arteriovenous fistula (AVF) and Parkes Weber Syndrome. The invention also relates to a pharmaceutical composition comprising at least one substance comprising a Ras antagonist or Ras activity modulator, as defined earlier and a physiologically acceptable carrier or excipient.

Preferably the pharmaceutical composition comprises at least one substance chosen from the group of compounds that are:
1) Ras inhibitors, such as ISIS2503, farnesyl transferase inhibitors R115777, SCH66336 and BMS 214662;
2) compounds inhibiting the downstream effector Raf, such as ISIS 5132 or BAY 43-9006; or
3) compounds inhibiting MEK, such as C1-1040 (also known as: PD184352).

The invention also relates to a medicament for treating, preventing or alleviating vascular anomalies comprising at least one substance as defined earlier in an effective amount for inactivating GTP bound Ras protein, preferably said substance is a Ras antagonist or Ras activity modulator.

The invention also relates to a method of treatment, prevention or alleviation of vascular anomalies comprising administering to a mammal in need of such treatment, prevention or alleviation a therapeutically effective amount of a substance that inactivates GTP bound Ras protein in said mammal.

The invention now being generally described may be more clearly understood by reference to the following examples, which are included merely for purposes of illustration of certain aspects and embodiments of the present invention and are not intended to limit the invention.

### DESCRIPTION OF FIGURES

FIG. 1. Clinical and histological characteristics of capillary malformation (CM). (A and B) CM on face, subject I-2 (family M) and subject III-11 (family C), respectively. Subject III-11 (family C) had also an intramaxillar arteriovenous malformation. (C) CM on hand, subject III-1 (family A). (D) CM on thorax, subject III-4 (family C). (E), retroauricular CM, subject III-1 (family F). (F) hematoxylin eosin staining of CM. Asterisks (*) indicate dilated capillary-like channels within papillary dermis.

FIG. 2. Pedigrees of 13 families with inherited capillary malformation. Incomplete penetrance in families D, F, L and M. Numbered individuals participated in study.

FIG. 3. FIG. 3 Multipoint linkage analysis of chromosome 5q on 13 families. Thick line, multipoint Z-score; thin line, multipoint HLOD score with 90% penetrance and 0.3% phenocopy rate. Maximum multipoint Z-score of 6.72 obtained at marker AFM205WG7, and maximum multipoint HLOD score of 4.84 1 cM centromeric of AFM205WG7. Genetic distance, in cM, from 5pter shown below.

FIG. 4. Multipoint linkage analysis on nine families (A, C, D, F, H, I, J, K and L) linked to CMC1-locus. Analysis performed under 90% penetrance and 0.3% phenocopy rate. Maximum LOD score of 7.22 obtained at marker D5S2044 (1 cM centromeric of marker AFM205WG7). Most likely linked region located between markers D5S1962 and D5S652. Genetic distance, in cM, from 5pter shown below. Capillary malformation locus maps to 5q.

FIG. 5. Photographs of vascular malformations linked to RASA1 mutations. Individual numbers refer to pedigrees in FIG. 6. Atypical small round-to-oval shaped CMs (CM45 III-8, III-15 and IV-2, and CM8 II-6); large cutaneous CM (CM8 III-4); CM20 III-1 with a circumscribed AVM of the nose with a cutanequs capillary blush (arrow) and PW1 III-1 with Parkes Weber syndrome involving the lower limb (arrow).

FIG. 6. Pedigrees, vascular phenotypes and co-segregation of the identified mutations. Pedigrees are shown in the order of occurrence of the corresponding mutation in the gene. Numbered individuals were screened for mutations. In families PW1, CM45, CM20 and CM41 co-segregation of deletional mutations with the phenotype was performed on radioactive size-difference gel-electrophoresis. In family CM8, presence of RASA1^{1454(C>T)} was detected by Sau3A1 restriction digestion. The mutation destroys a Sau3A1 restriction enzyme cutting site that splits the 223 bp wild-type allele into 134 bp and 89 bp fragments. ?=phenotype unknown. Numbers on right upper corner refer to number of CM in affected individual. RASA1 Δ^{CT593-594} and RASA1 Δ^{GC2454-2455} were de novo mutations. Altogether four carriers were identified.

FIG. 7a. Schematic presentation of identified RASA1 mutations. Four deletional mutations RASA1 Δ^{CT593-594} RASA1 Δ^{T630}, RASA1 Δ^{GTCT1697-1700} and RASA1 Δ^{GC2454-2455} cause frame shifts and subsequent premature stop codons. Resulting hypothetical proteins are illustrated. RASA1^{1737(G>A)} results in an Cys-540-Tyr substitution in the pleckstrin homology domain at position 540. SH2, Src homology-2 domain; SH3, Src homology-3 domain; PH, pleckstrin homology domain; C2, protein kinase conserved region two; RASGAP, ras GTPase activating domain. Altered amino acid sequence due to frame shift is hatched.

FIG. 7b. Multiple alignment of pleckstrin domains.

Alignment of pleckstrin homology domain amino acid sequences around the C540Y mutation. Highly conserved phenylalanine, F, in bold. The mutated cysteine C540Y is boxed. Only 12/37 PH domains containing cysteine at this position are shown. The three orthologous PH domain sequences of RASGRF1 containing a tyrosine at this position, are also shown.

FIG. 8. RASA1 sequences.

cDNA sequence (SEQ ID NO 1) corresponding to the RASA1 gene and amino acid sequence (SEQ ID NO 2) representing RAS p21 activating protein 1, retrieved from http:/www.ncbi.nim.nih.gov/ under the accession number NM_{—}002890.1. The start codon (ATG) in the nucleic acid of SEQ ID NO 1 is at position 119.

TABLE 1

RASA1 Primers for amplification of exons and
small parts of the 5′ and 3′ introns

| | | |
|---|---|---|
| RAS-DF: | 5′ ACTGACAGGGGGAGGTACTG | SEQ ID NO 3 |
| RASDR: | 5′ TTCCCAAATTCCTGAACAGC | SEQ ID NO 4 |
| RASFF: | 5′ GGGAGCTGAAGGGGAGAC | SEQ ID NO 5 |
| RASFR: | 5′ CTACGCCAGCAGCAGTACCT | SEQ ID NO 6 |
| RAS2F: | 5′ AAGTGTCCATAGAAATTCTGCAT | SEQ ID NO 7 |
| RAS2R: | 5′ CATTTGGCTTCATAATAGGAATA | SEQ ID NO 8 |
| | AA | |
| RAS3F: | 5′ GGAAAAGAGTATGGAAATTATGG | SEQ ID NO 9 |
| | A | |
| RAS3R: | 5′ GCAATAGCTAAAACCATTATTGT | SEQ ID NO 10 |
| | ACTG | |
| RAS4F: | 5′ TGAATGATCCCATGGAGTTTTCT | SEQ ID NO 11 |
| RAS4R: | 5′ CGAAGTCTAGCTCTTTCAAATGC | SEQ ID NO 12 |
| RAS5F: | 5′ GGGTGTTTGACTCTAATTCCTTA | SEQ ID NO 12 |
| | CA | |
| RAS5R: | 5′ TCTGATTACGGACAAGATCCAA | SEQ ID NO 13 |
| RAS6F: | 5′ GTGTGGGGATATGTTTGCAG | SEQ ID NO 14 |
| RAS6R: | 5′ AAAAGTTAAGTCAGTCCAAAACC | SEQ ID NO 15 |
| | TT | |
| RAS7F: | 5′ CACTTTGAATTAAACTTACTATA | SEQ ID NO 17 |
| | TTGG | |
| RAS7R: | 5′ TGCTAAAGGCAAACACATGAT | SEQ ID NO 18 |
| RAS8F: | 5′ TGTTTATGACTTTGAATGCACTT | SEQ ID NO 19 |
| | TG | |
| RAS8R: | 5′ TTTTTGCGAAAAGTAAAAGATAG | SEQ ID NO 20 |
| | C | |
| RAS9F: | 5′ CCTTGGCAAGAAAGTTTACACA | SEQ ID NO 21 |
| RAS9R: | 5′ TGTGCAAAAACATACCACCA | SEQ ID NO 22 |
| RAS10F: | 5′ AGCGCTTTGGCTTTTAATTG | SEQ ID NO 23 |
| RAS10R: | 5′ TTCGGAGCTCCATATTTACAA | SEQ ID NO 24 |
| RAS11F: | 5′ GCTTTGGAATAAAAATTGATTG | SEQ ID NO 25 |
| | A | |
| RAS11R: | 5′ TTCCGAAAGAAAAATAGGAAAC | SEQ ID NO 26 |
| | C | |
| RAS12F: | 5′ TGAGTGTTTTGGAAGCTGGT | SEQ ID NO 27 |
| RAS12R: | 5′ TTTCAGGCGTTCTGTCACTTT | SEQ ID NO 28 |
| RAS13F: | 5′ GAAATGGCAGTCTAGAGAAGGA | SEQ ID NO 29 |
| | A | |
| RAS13R: | 5′ GCAAAGTGTTAGAGCAAAATGT | SEQ ID NO 30 |
| | G | |
| RAS14F: | 5′ TTTTGGCTTTGTATCTTAGAGT | SEQ ID NO 31 |
| | AATTG | |
| RAS14R: | 5′ ACAGAAAGAAATGCAATATGGT | SEQ ID NO 32 |
| RAS15F: | 5′ GCAGAAATAGGGGGTTTATTTG | SEQ ID NO 33 |
| RAS15R: | 5′ AAGACTTTCATTGTGAATTTTG | SEQ ID NO 34 |
| | AA | |
| RAS16&17F: | 5′ GGGAAGACTGAACACCAGGA | SEQ ID NO 35 |
| RAS16&17R: | 5′ TTCCAACAAAAACAAGACTGAT | SEQ ID NO 36 |
| RAS18F: | 5′ TTTCTTGTTAGTCTCATGGAGC | SEQ ID NO 37 |
| | A | |
| RAS18R: | 5′ AAACCCAGTTTCTTGTATCACA | SEQ ID NO 38 |
| | CTA | |
| RAS19F: | 5′ CCAATTTGGTCACATTAGGTCA | SEQ ID NO 39 |
| RAS19R: | 5′ TTTTCCTTAAAATGTAATTGGC | SEQ ID NO 40 |
| | TAC | |
| RAS20F: | 5′ CAACCTCGAAAACTATAACTAC | SEQ ID NO 41 |
| | TTG | |
| RAS20R: | 5′ AACAGAAAAGCTTTCACGTTTT | SEQ ID NO 42 |
| | A | |
| RAS21F: | 5′ TGGCTGCTAGGAGATCAGTG | SEQ ID NO 43 |
| RAS21R: | 5′ TGCAACAGGGCTTTGACATA | SEQ ID NO 44 |
| RAS22F: | 5′ TGGGTTCTATGAGTACTAAAAA | SEQ ID NO 45 |
| | TTC | |
| RAS22R: | 5′ TGACTAGAATTGGATGATCAAA | SEQ ID NO 46 |
| | AA | |
| RAS23F: | 5′ GGTTTAGCTGGAAGTGCTGTT | SEQ ID NO 47 |
| RAS23R: | 5′ TGGTTTTATCATGTCAAACTTG | SEQ ID NO 48 |
| | C | |
| RAS24F: | 5′ TTTGCACCAACCTAATAGATCA | SEQ ID NO 49 |
| | AA | |
| RAS24R: | 5′ GATTGCTGCTTAAATGGGTTA | SEQ ID NO 50 |
| RAS25F: | 5′ GGCAACAAGAGCGAAACTCT | SEQ ID NO 51 |
| RAS25R: | 5′ AAGTGTTATCTTTGAAACATCA | SEQ ID NO 52 |
| | TTG | |
| RAS26F: | 5′ TTCAAATCCAGGTTCCCATC | SEQ ID NO 53 |
| RAS26R: | 5′ GCTGAATCCATGCAGAACACT | SEQ ID NO 54 |
| RAS1CF: | 5′ GGACGAAGGTGACTCTCTGG | SEQ ID NO 55 |
| RAS1CR: | 5′ CAAACCACAGATGAAAAGGACA | SEQ ID NO 56 |
| RAS12Fmut: | 5′ GTTTTTATTTTAAAGGCCAAAC | SEQ ID NO 57 |
| | TA | |
| RAS12Fwt: | 5′ GTTTTTATTTTAAAGGCCAAAC | SEQ ID NO 58 |
| | TG | |
| RAS12Rmut: | 5′ TGCTGAACTACTATCTGAAAAT | SEQ ID NO 59 |
| RAS12Rwt: | 5′ TGCTGAACTACTATCTGAAAAC | SEQ ID NO 60 |
| Ras12BF: | 5′ CAG CTT CAA TCT GTT | SEQ ID NO 61 |
| | TGT AAC T | |

The notation “F” refers to forward primer; the notation “R” refers to reverse primers

### EXAMPLES

### Informed Consent

Informed consent was obtained from all subjects participating in the study, as approved by the ethics committee of the Medical Faculty, Universite catholique de Louvain, Belgium, Saitama Children's Medical Center, Canada, and Boston Children's hospital, USA.

### Example 1

### Identification of the 23 cm Locus for Familial Capillary Malformation

Patients

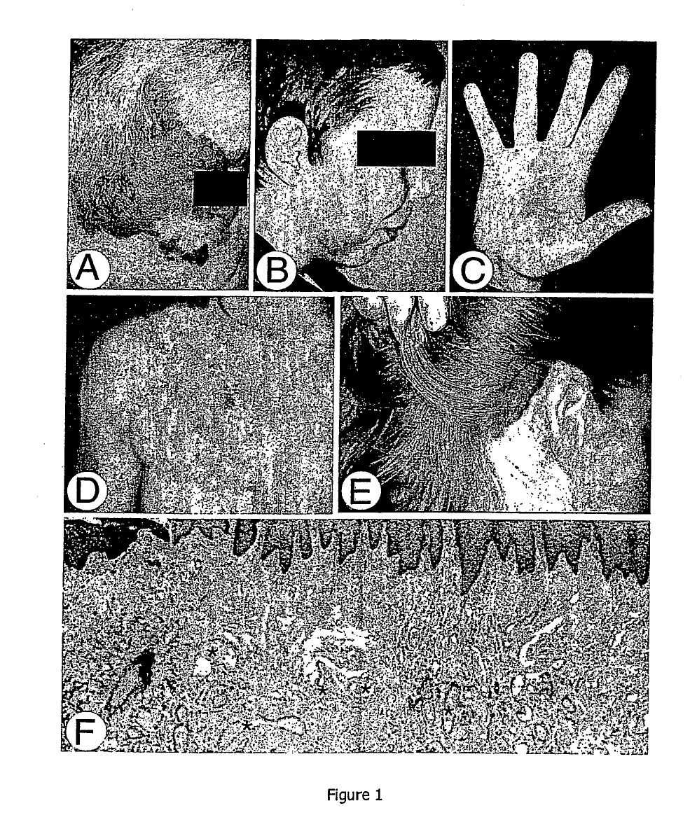

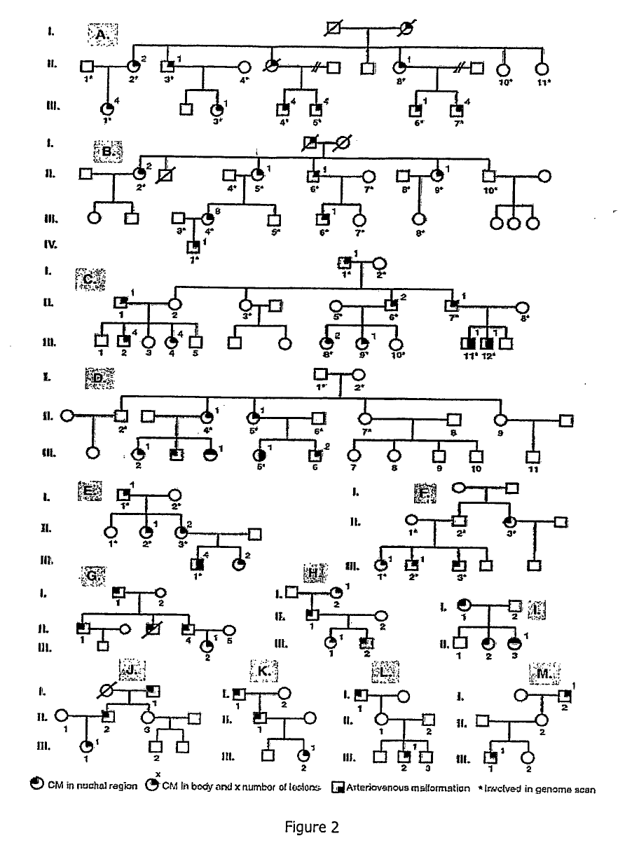

Blood or buccal brush samples were collected from 60 affected and 51 unaffected individuals (FIG. 2). Patients were clinically examined by a plastic surgeon (L M Boon, J B Mulliken and S Watanabe), or general practitioner (H Grynberg). In the 13 families involved in this study, most CMs were pink-to-purple macular lesions, measuring a few centimeters in diameter (FIG. 1). All subjects with a CM of at least 1 cm in diameter were considered affected. Individuals with only one lesion, smaller than 1 cm, or with faint nuchal stain, reminiscent of a fading birthmark, were considered to be unaffected. Out of 60 affected subjects, 19 had a lesion on the face, 15 in the nuchal region and 26 in other parts of the body. Fifteen subjects had multiple lesions (FIG. 2). In subject III-12, in family C, and subject III-1 in family E, an arteriovenous malformation underlay the cutaneous vascular stain (FIG. 2). Subject III-5, in family D, had an arteriovenous fistula between the left carotid artery and jugular vein, a cutaneous vascular stain and soft tissue hypertrophy of the homolateral face. Subject III-11, in family C, had a hemi-facial CM associated with left intramaxillar arteriovenous malformation (FIG. 1B).

Methods

Genomic DNA was extracted from blood samples using DNA purification kit (Westburg, the Netherlands) or from buccal cells using a lysis method, as described (Richards et al., 1993). The six most informative families (A-F) were selected for a genome scan. Due to space constraints on acrylamide gels, some unaffected individuals were left out and the screening was performed on 34 affected and 26 unaffected subjects (FIG. 2). Since none of the six families showed evidence of sex-linked inheritance, the genome scan was restricted to the autosomes. Fluorescently labeled polymorphic markers from Human MapPairs genome wide screening set (n=356, 10 cM average resolution) were amplified by PCR using the conditions recommended by the supplier (LI-COR, Westburg, the Netherlands). Amplified markers were electrophoresed on 6.5% acrylamide gels on Gene Reader 4200 DNA analyser, and genotyped with SAGA GT 2.0 software (LI-COR, Westburg, the Netherlands). Altogether, 168 additional markers, synthesized by Gibco Lifetechnologies (UK) or Isogen (the Netherlands), were used to cover genomic regions where Human MapPairs markers were uninformative. These markers were radioactively end-labeled with γ-[32P] using polynucleotide kinase (TAKARA/Bio Whittaker, Belgium) before amplification by PCR, and electrophoresed on 5% acrylamide gels, and scored manually after autoradiography overnight. Multipoint linkage analyses were performed with Genehunter 2.0 (Kruglyak et al., 1996). The unaffected grandparents in family D(I-1 and I-2) and unaffected subject II-2 in family F were considered unknown for CM phenotype in all linkage calculations (FIG. 2).

Results

CM segregated as a dominant trait in the 13 studied families. Evidence for incomplete penetrance was noted in families D, F, L and M (FIG. 2). In addition, phenotypic variation from single small CM in extremities to large facial lesion with arteriovenous involvement, was observed (FIGS. 1 and 2).

A non-parametric multipoint linkage analysis was performed first. This identified strong evidence of linkage between CMs and chromosome 5q. A maximum Z-score of 4.50 with a P-value of 0.0025 was found between markers D5S401 and D5S2044, and a 28 cM region with a P-value<0.01 was observed between markers D5S357 and D5S652. Suggestive evidence of linkage (P-value<0.05) was also found on chromosomes 2p (P=0.031), 4q (P=0.049), 6q (P=0.015), 7q (P=0.045), 8p (P=0.045), 10q (P=0.045) and 12p (P=0.028).

Genome wide multipoint linkage analysis was then performed under the assumption of autosomal dominant mode of inheritance with an allelic frequency of 0.0001 for the disease. The analysis was carried out with 90 and 80% penetrances, and the phenocopy rate was set at 0.3%, corresponding to the incidence of CM in the general population. With 90% penetrance, a statistically significant multipoint HLOD of 4.58 (α-value 0.92) was obtained on 5q between markers D5S357 and D5S2003, confirming the results of non-parametric analysis. There was also suggestive evidence of linkage (HLOD>1.0) on 6q, with a multipoint HLOD score of 1.06 (α-value 0.25). No other chromosomes exhibited evidence of linkage. 5q and 6q also gave the highest multipoint HLOD scores under 80% penetrance: 4.41 and 0.98, respectively.

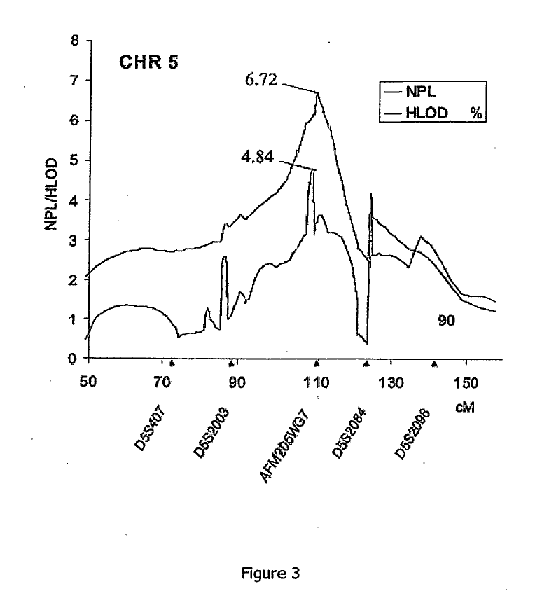

In order to further define the linked region on chromosome 5q, 27 additional markers were genotyped for the six families, including the family members, mostly unaffected, who were excluded in the initial screening (FIG. 2). Furthermore, seven additional small CM families (FIG. 2) were genotyped with eight markers on chromosome 5q. Non-parametric linkage analysis using these 13 families yielded a maximum Z-score of 6.72 (P-value=0.000136) at marker AFM205WG7 on 5q15 (FIG. 3). The Z-score remained significant (P<0.01) over a 69 cM region between D5S407 and D5S2098, with the exemption of an interval of 1 cM (proximal to marker D5S2084) (P=0.011).

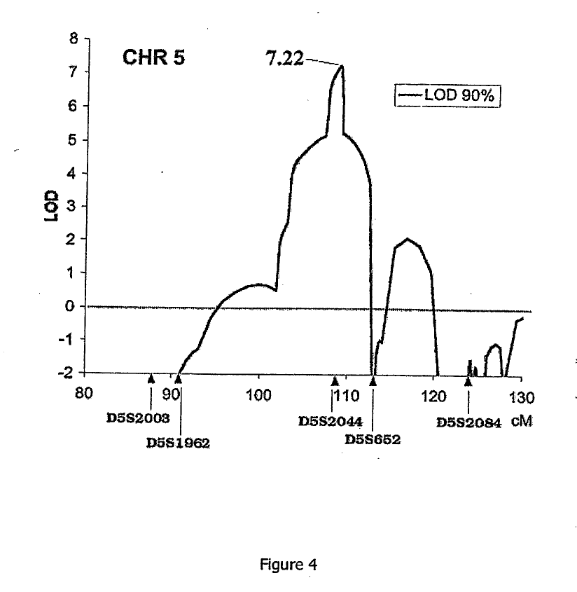

Parametric multipoint linkage analyses of chromosome 5q, under various penetrances (50-90%) with all the 13 families, gave the highest multipoint HLOD scores with 90% penetrance. A maximum HLOD of 4.84 (α-value 0.67) was obtained at marker D5S2044, which is 1 cM centromeric of marker AFM205WG7 that yielded the peak in the NPL analysis (FIG. 3). Another peak of HLOD>3.0 was 4.09 (α-value 0.51), between markers D5S2084 and D5S1453. In the studied 5q region, the estimated fraction of families linked (α-values) varied between 0.51 and 0.67, suggesting genetic heterogeneity. When the families B, E, G and M, which yielded negative multipoint LOD scores at marker D5S2044, were excluded from linkage analysis, a maximum multipoint LOD score of 7.22 (α-value 1.00) was obtained at marker D5S2044 using 90% penetrance (FIG. 4). The most likely linked region, defined by borders of multipoint LOD score<−2.00, was between markers D5S1962 and D5S652, covering 23 cM (FIG. 4).

### Example 2

### Reduction of the Susceptible Region to the CMC-1 Locus

Patients

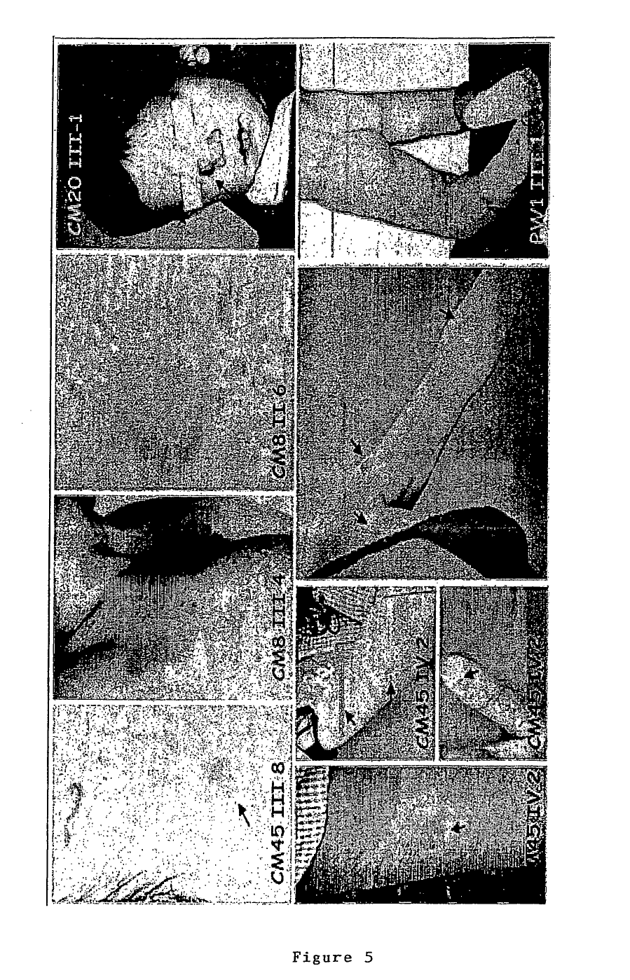

Families CM8, CM11 and CM20 have been reported earlier (Eerola et al., 2002) and are the same families as families C, D and E of Example 1. The atypical CMs were multiple small (1-2 cm in diameter) round-to-oval and pinkish-red in color. CM-associated vascular anomalies and tissue hypertrophy characterized the following phenotypes. In family PW1, subject III-1 had Parkes Weber syndrome (FIG. 5) and subject III-2 had an intracranial AVM as well as multiple cutaneous CMs. In family CM45, subject III-15 had an intracranial AVM, and five cutaneous CMs of the extremities, and subject IV-11 had a cutaneous AVM of the ankle, and three cutaneous CMs located on the face, thorax and thigh. In family CM8, subject III-11 had a left intramaxillary AVM causing bony hypertrophy, and an extensive hemifacial CM with soft tissue hypertrophy, and subject III-12 had a CM of the mid lower lip with hypertrophy, and an intramandibular AVM causing dental distortion. In family CM20, subject III-1 had a deep facial AVM with an overlying cutaneous vascular stain (FIG. 5). In family CM41, subject III-10 had a cutaneous AVM of the forehead and three small CMs located on the back, shoulder and a fourth digit, and subject IV-10 had a stage I cutaneous AVM of the right fifth finger and five cutaneous CMs of the extremities, face and scrotum. In family CM11, subject III-5 had a facial capillary stain distal to an AVF between the left carotid artery and the jugular vein, causing cardiac overload, requiring medication since birth. There was also soft tissue hypertrophy of the involved face and a small CM on the left wrist. Four of the families carrying mutations were from Belgium, one from Canada and one from the USA.

Linkage to the CMC-1 locus was tested in family 45 with 17 polymorphic markers: D5S1962, D5S646, D5S1501, D5S641, D5S2029, D5S2094, D5S428, D5S459, D5S617, D5S2103, D5S1725, D5S401, D5S2044, D5S2100, GATA5F09, AFM205and D5S652, as described in Example 1 and in Eerola et al., 2002.

The susceptibility locus was narrowed to the CMC-1 locus of 5 cM, between markers D5S459 and GATA5F09.

### Example 3

### Analysis of the Mutations in the CMC-1 Locus

SSCP and Heteroduplex Analyses

The genomic sequences containing the RASA1 gene were identified by a blast-homology search with the RASA1 mRNA sequence (NM_{—}002890.1) on the human genome sequence at the NCBI blast server. Homo sapiens chromosome 5 working draft sequence NT_{—}037660.1 containing the gene was retrieved from the entrez database. 27 sets of primers (represented by SEQ ID NOs 3 to 54, Table 1) were designed to amplify all the 25 exons including exon-intron boundaries. The isoform 2-specific exon 1 was also screened.

The primers as represented in Table 1 were used as pairs to amplify fragments of the RASA1 gene, as follows: RAS1DF/RAS1DR, RAS1FF/RAS1FR, RAS2F/RAS2R, RAS3F/RAS3R . . . The first exon was amplified in two separate fragments using primer pairs RAS1DF/RAS1DR and RAS1FF/RAS1FR, and exons 16 and 17 were amplified in a single amplicon using primer pair RAS16&17F/RAS16&17R. All the other exons were amplified in a single amplicon, using the corresponding primer pair. An additional primer pair was created for the alternatively spliced exon 1, called exon 1C (primers RAS1CF and RAS1CR, represented by SEQ ID NO 55 and 56, respectively).

PCR reactions, subsequent SSCP and heteroduplex analyses and direct sequencing were performed, as described earlier (Brouillard et al., 2002).

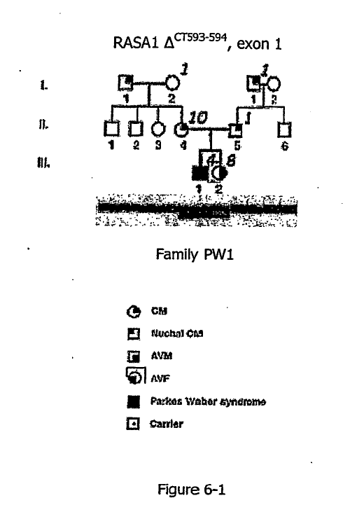

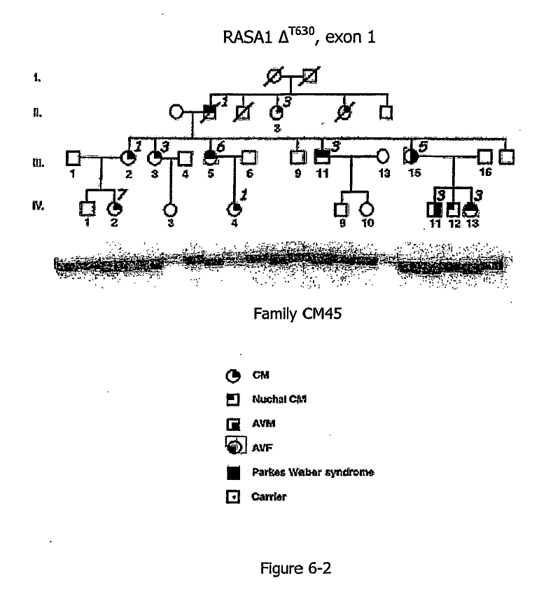

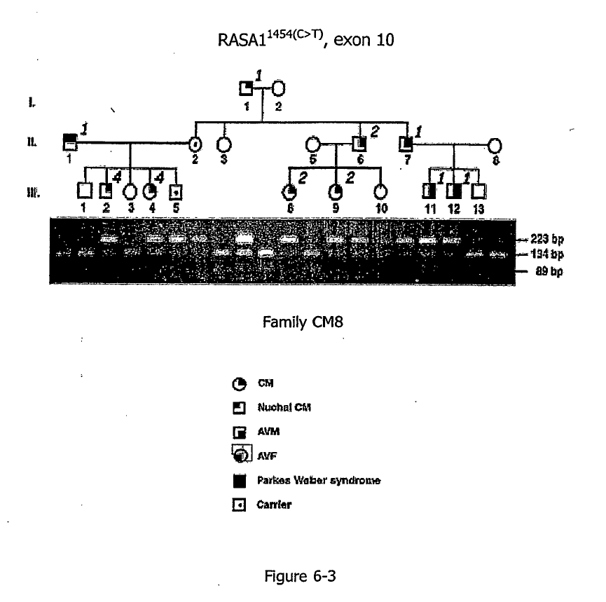

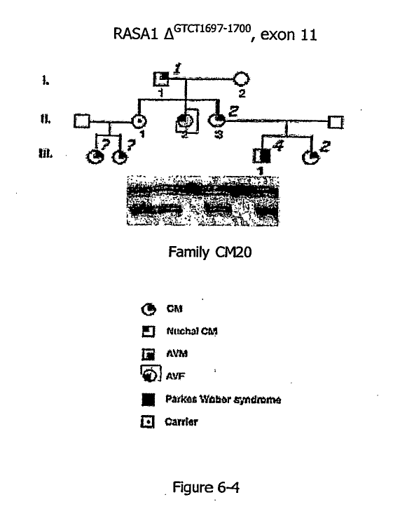

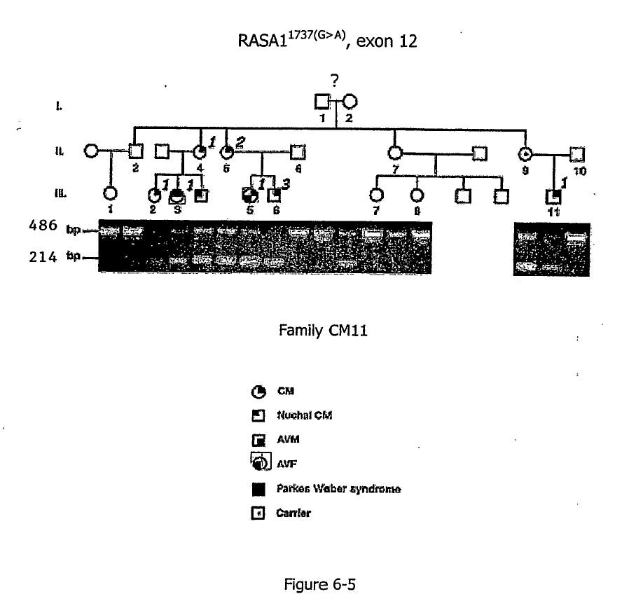

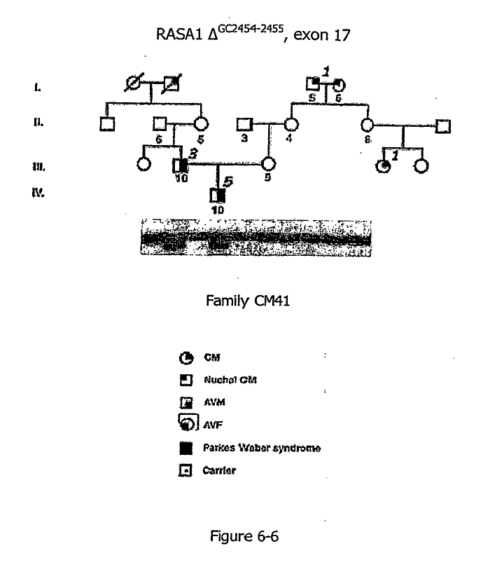

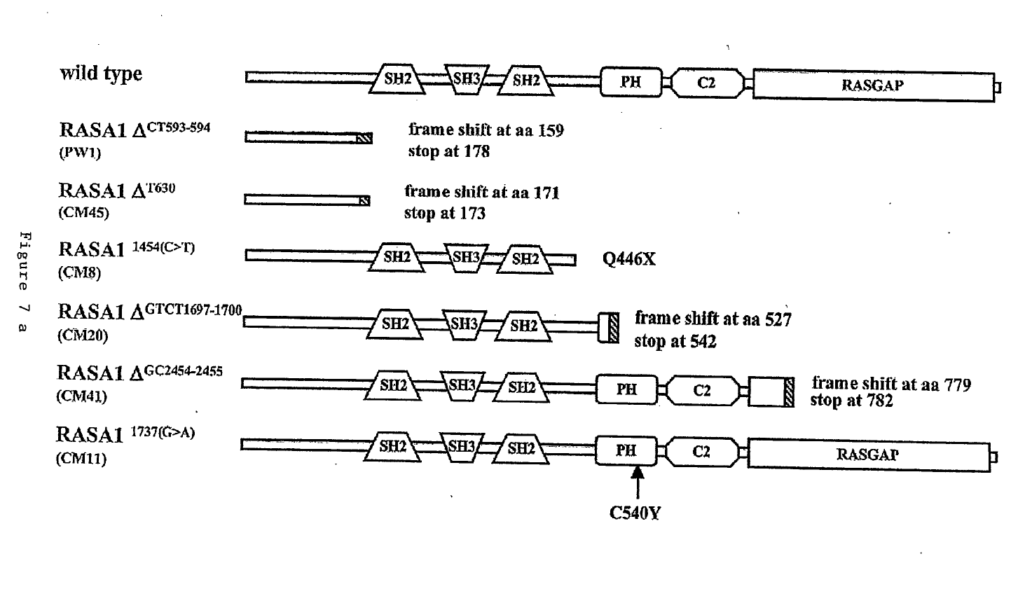

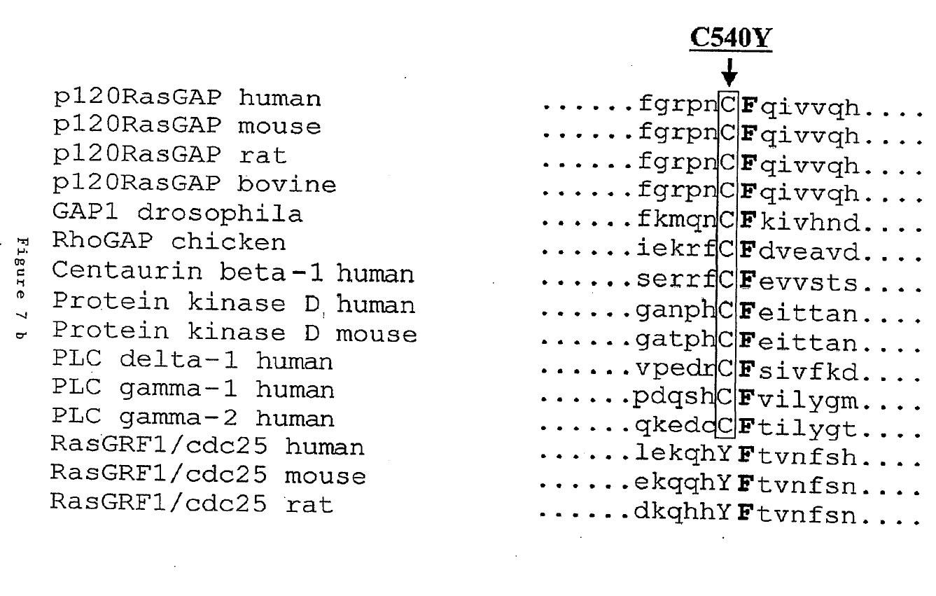

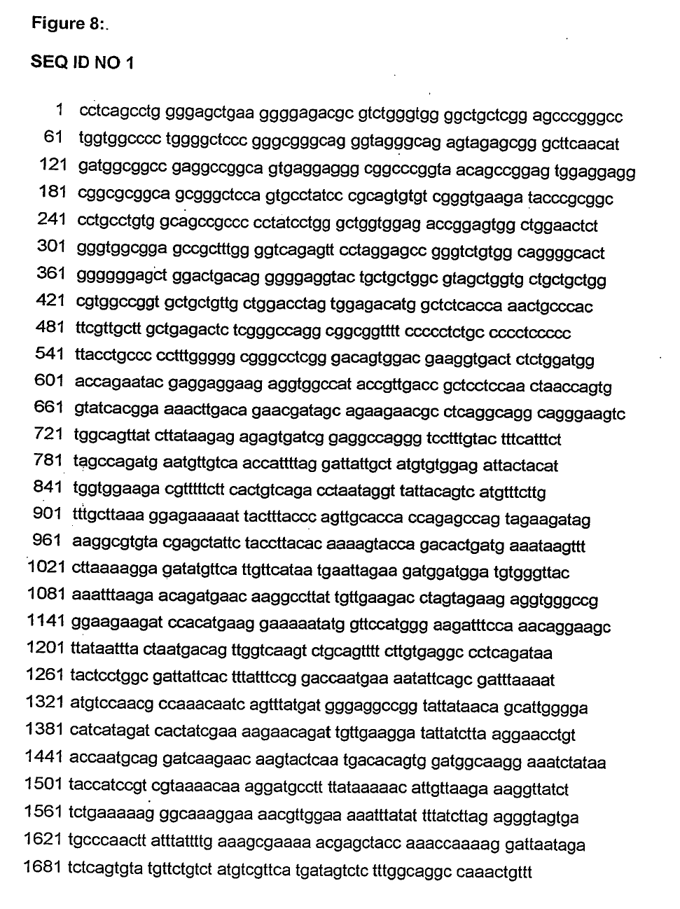

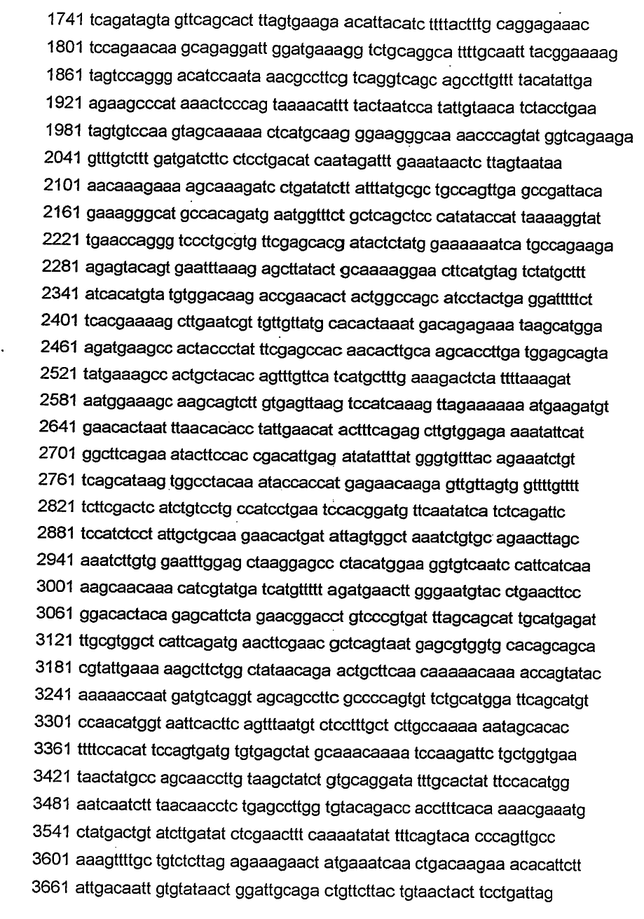

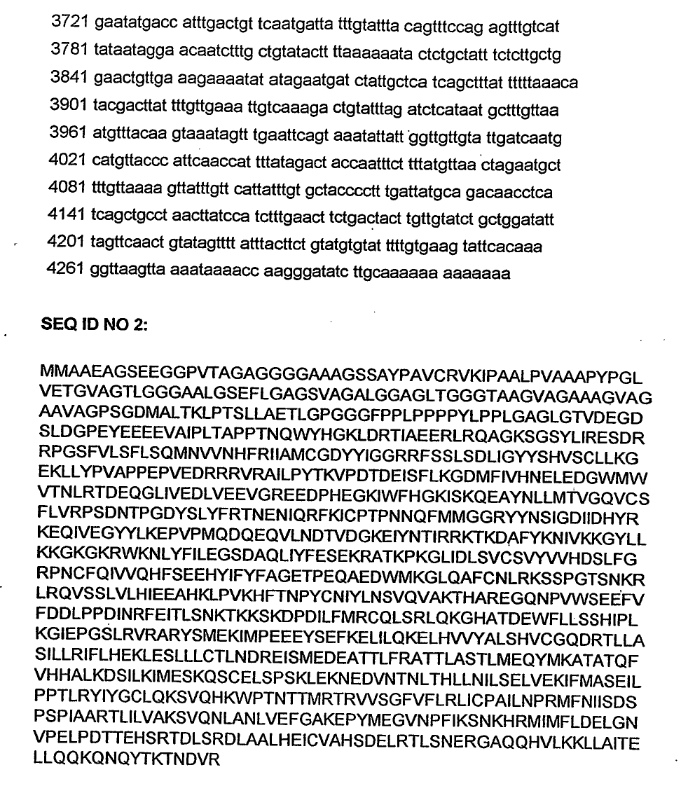

Seventeen families manifesting predominantly cutaneous CM were subjected to RASA1 screening for mutations using SSCP and heteroduplex analyses of all the 26 exons and exon-intron boundaries of the gene, e.g. 25 exons and the isoform 2-specific exon 1. RASA1 mutations were found in six families. None of the mutations was identified in fifty healthy controls. The alterations included four deletions in the coding region, RASA1 Δ^{CT593-594}, RASA1 Δ^{GTCT1697-1700}, RASA1 Δ^{GC2454-2455} and RASA1 Δ^{T630}, and two substitutions, RASA1^{1454(C>T)} leading to a nonsense mutation Q446X and RASA1^{1737(G>A)} resulting in Cys-to-Tyr substitution at the amino acid 540 (C540Y) (FIG. 7a). The deletions led to reading frame shifts and subsequent premature stop codons, predicted to result in a truncated protein (FIG. 7a). The only amino acid substitution, C540Y, occurred in the pleckstrin homology (PH) domain (FIG. 7b), which has been shown to regulate interaction between p120-RasGap and RAS (Drugan et al., 2000). The adjacent amino acid phenylalanine (541) is highly conserved in PH domains (FIG. 7b). Cysteine is present at position 540 in 37/201 (15%) of the PH domains in Prosite database, whereas tyrosine is only found in the PH domains of guanine nucleotide releasing protein (RasGRF1/CDC25) of three species (FIG. 7b). Interestingly, the PH domain of RasGRF1/CDC25 did not inhibit Ras-induced transformation like the PH domain of p120RasGAP in overexpression experiment on NIH 3T3 cells (Drugan et al., 2000). The RASA1^{1737(G>A)} mutation most likely results in a functionless p120-RasGap. Two of the six mutations occurred de novo (FIG. 6). A further mutation which was identified is in the splice site of exon 17: IVS17+1delG: RASA1Δ^{GIVS17+1}.

Mutations co-segregated with, vascular malformations in all six families (FIG. 6). The affected individuals exhibited mostly atypical CMs, characterized as multiple, small (1-2 cm in diameter) pink-to-red circular lesions (FIG. 5). In each family, at least one individual had either an AVM or an AVF concurrently with CMs. In addition, two patients were diagnosed as Parkes Weber syndrome with multiple AVFs, and soft and skeletal tissue hypertrophy of the affected limb. Another patient had overgrowth of the soft tissue of the face, in association with an uncommon AVF between the ipsilateral carotid artery and the jugular vein. Four individuals who had no obvious vascular malformation carried a mutation, giving an overall penetrance of 89%. The identification of atypical round-to-oval CMs and their association with high-flow arterial lesions constitutes a heretofore undescribed clinical and genetic entity.

Co-segregation Analysis

The fragments, in which deletional mutations were detected, were amplified by radioactive PCR from genomic DNA from all family members, and analysed on size difference gel-electrophoresis to detect co-segregation (Families PW1, CM20, CM41 and CM45, FIG. 2). Co-segregation of the mutation RASA1^{1454(C>T)} (Q446X), which destroys a Sau3A1 restriction enzyme cutting site, was screened by Sau3A1 (Promega) digestion of exon 10 genomic amplicons of all individuals in family CM8 (FIG. 6), in conditions recommended. Mutation RASA1^{1737(G>A)} (C540Y) in family CM11 did not change any restriction enzyme cutting site (FIG. 6). Thus, it was screened by allele specific PCR on both strands with the following primer pairs:

| | |
|---|---|
| RAS12BF/RASRwt | Coding strand mutant (317 bp) |
| | (not shown in photograph) |
| RAS12BF/RAS12Rmut | Coding strand wild-type (317 bp) |
| | (not shown in photograph) |
| RAS12Fwt/RAS12R | Reverse strand mutant (214 bp) |
| RAS12Fmut/RAS12R | Reverse strand wild-type (214 bp) |
| RAS12BF/RAS12R | Control (functions irrespective of |
| | mutation) (486 bp) |

Since the PCR reaction is allele specific, the mutant primer will only work if the DNA to be tested contains a mutation, otherwise, only the wild-type primer pair will work. The two tests, one with the wild type primer pair and another with the mutant primer pair. FIG. 6-5 (family CM 11) shows the result for the mutant primer pair and the wild type control PCR (486 bp).

The sequence of the primers is shown in Table 1. Fifty unrelated healthy controls were also screened for all mutations.

Electronic Data Base Information

Pleckstrin domain homology searches were performed using the Prosite database at http://us.expasy.org/prosite/. NM_{—}002890.1 and NT_{—}037660.1 nucleotide sequences for RASA1 were retrieved from the entrez database at http://www.ncbi.nlm.nih.gov/entrez/. The online version of “Mendelian Inheritance in Man” was used at http://www3.ncbi.nlm.nih.gov/omim/. Genomic sequences for RASA1 were identified using the NCBI Blast server at http://ncbi.nim.nhi.gov/.

### REFERENCES

Adjei A A. (2001) Ras signaling pathway proteins as therapeutic targets Curr. Pharm. Des Nov. 7(16), 1581-94.

Barsky, S. H., Rosen, S., Geer, D. E. & Noe, J. M. (1980) The nature and evolution of port wine stains: A computer-assisted study. J. Invest. Dermatol. 74, 154-157.

Bi, W., Drake, C. J., Schwartz, J. J. (1999) The transcription factor MEF2C-null mouse exhibits complex vascular malformations and reduced cardiac expression of angiopoietin 1 and VEGF. Dev. Biol. 15, 255-267.

Brouillard P. et al. (2002) Mutations in a novel factor, glomulin, are responsible for glomuvenous malformations (“glomangiomas”). Am. J. Hum. Genet. 70, 866-874.

Dancey J E, (2002) Agents targeting ras signaling pathway. Curr. Pharm. Des. 8 (25): 2259-67

Denhardt, D. T., (1996) Biochem. J. 310, 729-747

Drugan, J. K., Rogers-Graham, K., Gilmer, T., Campbell, S. & Clark, G. J. (2000) The Ras/p120 GTPase-activating protein (GAP) interaction is regulated by the p120 GAP pleckstrin homology domain. J. Biol. Chem. 275, 35021-35027.

Eerola et al., (2001) American Journal of Human Genetics, page 280.

Eerola, I. et al. (2002) Locus for susceptibility for familial capillary malformation (‘port-wine stain’) maps to 5q. Eur. J. Hum. Genet. 10, 375-380.

Hanahan and Weinberg (2000) Cell 57-70.

Henkemeyer, M. et al. (1995) Vascular system defects and neuronal apoptosis in mice lacking ras GTPase-activating protein. Nature 377, 695-701.

Herrera R. and Sebolt-Leopold J. S. (2002) Unraveling the complexities of the Raf/MAP kinase pathway for pharmacological intervention. Trends in Molecular Medicine vol 8, no 4, (suppl.) S27-31:

Kruglyak L., Daly, M. J., Reeve-Daly M. P & Lander E. S. (1996) Parametric and nonparametric kinkage analysis: a unified multipoint approach. Am. J. Hum. Genet. 58, 1347-1363.

Mulliken, J. B. & Glowacki, J. (1982) Hemangiomas and vascular malformation in infants and children: A classification based on endothelial characteristics. Plast. Reconstr. Surg. 69, 412-420.

Mulliken, J. B. and Young, A. E. (1988). Vascular birthmarks: Hemangiomas and Malformations, W B Saunders, Philadelphia.

Richards, B., Skoletsky, J., Shuber A. P. et al. (1993) Multiplex PCT amplification from the CFTR gene using DNA prepared from buccal brushes/swabs. Hum. Mol. Genet. 2, 159-163.

Vikkula, M., Boon, L. M., Mulliken, J. B., Olsen B. R. (1998) Molecular basis of vascular anomalies. Trends Cardiovasc. Med. 8: 281-292.

1. A method of treating, preventing or alleviating vascular anomalies comprising administering a therapeutically effective amount of a substance able to convert active GTP bound Ras protein into inactive GDP bound Ras protein intracellularly in a cell to a mammal in need thereof.

2. A method of treating, preventing or alleviating vascular anomalies comprising administering a therapeutically effective amount of a substance that converts active GTP bound Ras protein into inactive GDP bound Ras protein in a cell to a mammal in need thereof.

3. The method according to claim 1, wherein said substance modulates the status of p120 RasGAP in a cell resulting in the presence of p120GAP protein in said cell in an amount effective to inactivate GTP bound Ras protein.

4. The method according to claim 1, wherein said substance is a Ras antagonist or Ras activity modulator.

5. The method according to claim 1, wherein said substance comprises:
(i) Ras inhibitors, selected from the group consisting of ISIS2503, farnesyl transferase inhibitors R115777, SCH66336, and BMS 214662;
(ii) compounds inhibiting the downstream effector Raf, selected from the group consisting of ISIS 5132 and BAY 43-9006; or
(iii) a compounds inhibiting MEK, which is C1-1040.

6. The method according to claim 1, wherein said vascular anomalies are selected from the group consisting of capillary malformations (CM), arteriovenous malformations (AVM), arteriovenous fistulas (AVF), and Parkes Weber Syndrome.

7. A pharmaceutical composition comprising at least one substance as defined in claim 1 and a physiologically acceptable carrier or excipient.

8. The pharmaceutical composition according to claim 7, wherein said substance is a Ras antagonist or Ras activity modulator.

9. A medicament for treating, preventing or alleviating vascular anomalies comprising at least one substance as defined in claim 1 in an effective amount for inactivating GTP bound Ras protein.

10. The medicament according to claim 9 wherein said substance is a Ras antagonist or a Ras activity modulator.

11. A method of treatment, prevention or alleviation of vascular anomalies comprising administering to a mammal in need of such treatment, prevention or alleviation a therapeutically effective amount of a substance that inactivates GTP bound Ras protein in said mammal.

12. The method according to claim 11 wherein said substance is an Ras antagonist or Ras activity modulator.

13. A method for diagnosis of inherited capillary malformation using a nucleic acid of at least 10 nucleotides having a sequence which is substantially complementary to a sequence in the RASA1 gene.

14. A probe for in vitro diagnosis of vascular anomalies in a subject carrying a mutation in the RASA1 gene, wherein said probe comprises a sequence of at least about 10 successive nucleotides substantially complementary to a sequence in the RASA1 gene wherein one of the following deletions or mutations occurs: RASA1Δ^{CT593-594}, RASA1Δ^{GTCT1697-1700}, RASA1Δ^{GC2454-2455}, RASA1Δ^{T630}, RASA1^{1454(C>T)}, RASA1Δ^{GIVS17+1}, or RASA1Δ^{1737(G>A)}.

15. A method for in vitro diagnosis of vascular anomalies using the probe of claim 14.

16. A method for in vitro diagnosis of vascular anomalies in a subject carrying a mutation in the RASA1 gene, said process comprising:
contacting DNA, isolated from a biological sample taken from a patient, with the probe of claim 14, with said contact being carried out under conditions enabling the formation of hybridization complexes between said probe and said DNA;
detecting hybridization complexes which have been formed; and
detecting a mutation in the RASA1 gene in at least one of the positions RASA1Δ^{CT593-594}, RASA1Δ^{GTCT1697-1700}, RASA1Δ^{GC2454-2455}, RASA1Δ^{T630}, RASA1^{1454(C>T)}, RASA1Δ^{GIVS17+1}, or RASA1Δ^{1737(G>A)} whereby the vascular anomaly is diagnosed.

17. The method according to claim 13, wherein said probe is detectably labeled.

18. A method for diagnosis of inherited capillary malformation using at least one nucleic acid substantially complementary to a sequence in the RASA1 gene, said sequence flanking the region wherein a mutation may occur.

19. A primer for in vitro diagnosis of vascular anomalies in a subject carrying a mutation in the RASA1 gene, wherein said primer comprises a sequence of from about 10 successive nucleotides specifically amplifying the sequence of the RASA1 gene wherein one of the following deletions or mutations occurs: RASA1Δ^{CT593-594}, RASA1Δ^{GTCT1697-1700}, RASA1Δ^{GC2454-2455}, RASA1Δ^{T630}, RASA1^{1454(C>T)}, RASA1Δ^{GIV517-301}, or RASA1Δ^{1737(G>A)}.

20. A primer comprising the sequence of any of SEQ ID NOs 3 to 61.

21. A method for in vitro diagnosis of vascular anomalies using the primer of claim 19.

22. A method according to claim 11, wherein said vascular anomaly is selected from the group consisting of capillary malformation (CM), arteriovenous malformation (AVM), arteriovenous fistula (AVF), and Parkes Weber Syndrome.

23. A method for in vitro diagnosis of inherited vascular malformations which comprises the use of a nucleic acid of at least 10 contiguous basepairs chosen from the sequence of the RASA1 gene.

24. A kit for in vitro diagnosis of vascular anomalies in a subject carrying a mutation in the RASA1 gene, said kit comprising one or more of the following:
a determined amount of a nucleotide probe of claim 14,
primers to amplify a fragment of chromosome 5 comprising at least part of the RASA1 gene,
an appropriate medium for creating an hybridization reaction between the fragment and the probe,
reagents enabling the detection of hybridization complexes which have been formed between the fragment and the probe during any hybridization reaction.

25. A kit for in vitro diagnosis of vascular anomalies in a subject carrying a mutation in the RASA1 gene, said kit comprising at least one primer selected from the group consisting of SEQ ID NOs 3 to 61.

26. A kit for the detection of genetic deletions or mutations associated with at least one condition selected from the group consisting of CM, AVM, AVF, and Parkes Weber Syndrome comprising at least one probe of claim 14 or.

27. A method of treating, preventing or alleviating vascular anomalies comprising administering a therapeutic amount of a Ras antagonist or a Ras activity modulator to a mammal in need thereof.

28. (canceled)

29. A method for in vitro diagnosis of vascular anomalies using the primer of claim 20.

30. A kit for the detection of genetic deletions or mutations associated with at least one condition selected from the group consisting of CM, AVM, AVF, and Parkes Weber Syndrome comprising at least one primer of claim 19.


SEQUENCE LISTING (60 sequences)

SEQ ID NO: 1 - DNA, 4307 bp, Homo sapiens
cctcagcctggggagctgaaggggagacgcgtctgggtggggctgctcggagcccgggcctggtggcccctggggctcccgggcgggcagggtagggcagagtagagcgggcttcaacatgatggcggccgaggccggcagtgaggagggcggcccggtaacagccggagctggaggaggcggcgcggcagcgggctccagtgcctatcccgcagtgtgtcgggtgaagatacccgcggccctgcctgtggcagccgccccctatcctgggctggtggagaccggagtggctggaactctgggtggcggagccgctttggggtcagagttcctaggagccgggtctgtggcaggggcactggggggagctggactgacagggggaggtactgctgctggcgtagctggtgctgctgctggcgtggccggtgctgctgttgctggacctagtggagacatggctctcaccaaactgcccacttcgttgcttgctgagactctcgggccaggcggcggttttccccctctgccccctcccccttacctgccccctttgggggcgggcctcgggacagtggacgaaggtgactctctggatggaccagaatacgaggaggaagaggtggccataccgttgaccgctcctccaactaaccagtggtatcacggaaaacttgacagaacgatagcagaagaacgcctcaggcaggcagggaagtctggcagttatcttataagagagagtgatcggaggccagggtcctttgtactttcatttcttagccagatgaatgttgtcaaccattttaggattattgctatgtgtggagattactacattggtggaagacgtttttcttcactgtcagacctaataggttattacagtcatgtttcttgtttgcttaaaggagaaaaattactttacccagttgcaccaccagagccagtagaagatagaaggcgtgtacgagctattctaccttacacaaaagtaccagacactgatgaaataagtttcttaaaaggagatatgttcattgttcataatgaattagaagatggatggatgtgggttacaaatttaagaacagatgaacaaggccttattgttgaagacctagtagaagaggtgggccgggaagaagatccacatgaaggaaaaatatggttccatgggaagatttccaaacaggaagcttataatttactaatgacagttggtcaagtctgcagttttcttgtgaggccctcagataatactcctggcgattattcactttatttccggaccaatgaaaatattcagcgatttaaaatatgtccaacgccaaacaatcagtttatgatgggaggccggtattataacagcattggggacatcatagatcactatcgaaaagaacagattgttgaaggatattatcttaaggaacctgtaccaatgcaggatcaagaacaagtactcaatgacacagtggatggcaaggaaatctataataccatccgtcgtaaaacaaaggatgccttttataaaaacattgttaagaaaggttatcttctgaaaaagggcaaaggaaaacgttggaaaaatttatattttatcttagagggtagtgatgcccaacttatttattttgaaagcgaaaaacgagctaccaaaccaaaaggattaatagatctcagtgtatgttctgtctatgtcgttcatgatagtctctttggcaggccaaactgttttcagatagtagttcagcactttagtgaagaacattacatcttttactttgcaggagaaactccagaacaagcagaggattggatgaaaggtctgcaggcattttgcaatttacggaaaagtagtccagggacatccaataaacgccttcgtcaggtcagcagccttgttttacatattgaagaagcccataaactcccagtaaaacattttactaatccatattgtaacatctacctgaatagtgtccaagtagcaaaaactcatgcaagggaagggcaaaacccagtatggtcagaagagtttgtctttgatgatcttcctcctgacatcaatagatttgaaataactcttagtaataaaacaaagaaaagcaaagatcctgatatcttatttatgcgctgccagttgagccgattacagaaagggcatgccacagatgaatggtttctgctcagctcccatataccattaaaaggtattgaaccagggtccctgcgtgttcgagcacgatactctatggaaaaaatcatgccagaagaagagtacagtgaatttaaagagcttatactgcaaaaggaacttcatgtagtctatgctttatcacatgtatgtggacaagaccgaacactactggccagcatcctactgaggatttttcttcacgaaaagcttgaatcgttgttgttatgcacactaaatgacagagaaataagcatggaagatgaagccactaccctatttcgagccacaacacttgcaagcaccttgatggagcagtatatgaaagccactgctacacagtttgttcatcatgctttgaaagactctattttaaagataatggaaagcaagcagtcttgtgagttaagtccatcaaagttagaaaaaaatgaagatgtgaacactaatttaacacacctattgaacatactttcagagcttgtggagaaaatattcatggcttcagaaatacttccaccgacattgagatatatttatgggtgtttacagaaatctgttcagcataagtggcctacaaataccaccatgagaacaagagttgttagtggttttgtttttcttcgactcatctgtcctgccatcctgaatccacggatgttcaatatcatctcagattctccatctcctattgctgcaagaacactgatattagtggctaaatctgtgcagaacttagcaaatcttgtggaatttggagctaaggagccctacatggaaggtgtcaatccattcatcaaaagcaacaaacatcgtatgatcatgtttttagatgaacttgggaatgtacctgaacttccggacactacagagcattctagaacggacctgtcccgtgatttagcagcattgcatgagatttgcgtggctcattcagatgaacttcgaacgctcagtaatgagcgtggtgcacagcagcacgtattgaaaaagcttctggctataacagaactgcttcaacaaaaacaaaaccagtatacaaaaaccaatgatgtcaggtagcagccttcgccccagtgttctgcatggattcagcatgtccaacatggtaattcacttcagtttaatgtctcctttgctcttgccaaaaaatagcacacttttccacattccagtgatgtgtgagctatgcaaacaaaatccaagattctgctggtgaataactatgccagcaaccttgtaagctatctgtgcaggatatttgcactatttccacatggaatcaatctttaacaacctctgagccttggtgtacagaccacctttcacaaaacgaaatgctatgactgtatcttgatatctcgaactttcaaaatatattttcagtacacccagttgccaaagttttgctgtctcttagagaaagaactatgaaatcaactgacaagaaacacattcttattgacaattgtgtataactggattgcagactgttcttactgtaactacttcctgattaggaatatgaccatttgactgttcaatgattatttgtatttacagtttccagagtttgtcattataataggaacaatctttgctgtatacttttaaaaaatactctgctatttctcttgctggaactgttgaaagaaaatatatagaatgatctattgctcatcagctttattttttaaacatacgacttattttgttgaaattgtcaaagactgtatttagatctcataatgctttgttaaatgtttacaagtaaatagtttgaattcagtaaatattattggttgttgtattgatcaatgcatgttacccattcaaccattttatagactaccaatttcttttatgttaactagaatgcttttgttaaaagttatttgttcattatttgtgctacccctttgattatgcagacaacctcatcagctgcctaacttatccatctttgaacttctgactacttgttgtatctgctggatatttagttcaactgtatagttttatttacttctgtatgtgtatttttgtgaagtattcacaaaggttaagttaaaataaaaccaagggatatcttgcaaaaaaaaaaaaa

SEQ ID NO: 2 - protein, 1047 aa, Homo sapiens
MMAAEAGSEEGGPVTAGAGGGGAAAGSSAYPAVCRVKIPAALPVAAAPYPGLVETGVAGTLGGGAALGSEFLGAGSVAGALGGAGLTGGGTAAGVAGAAAGVAGAAVAGPSGDMALTKLPTSLLAETLGPGGGFPPLPPPPYLPPLGAGLGTVDEGDSLDGPEYEEEEVAIPLTAPPTNQWYHGKLDRTIAEERLRQAGKSGSYLIRESDRRPGSFVLSFLSQMNVVNHFRIIAMCGDYYIGGRRFSSLSDLIGYYSHVSCLLKGEKLLYPVAPPEPVEDRRRVRAILPYTKVPDTDEISFLKGDMFIVHNELEDGWMWVTNLRTDEQGLIVEDLVEEVGREEDPHEGKIWFHGKISKQEAYNLLMTVGQVCSFLVRPSDNTPGDYSLYFRTNENIQRFKICPTPNNQFMMGGRYYNSIGDIIDHYRKEQIVEGYYLKEPVPMQDQEQVLNDTVDGKEIYNTIRRKTKDAFYKNIVKKGYLLKKGKGKRWKNLYFILEGSDAQLIYFESEKRATKPKGLIDLSVCSVYVVHDSLFGRPNCFQIVVQHFSEEHYIFYFAGETPEQAEDWMKGLQAFCNLRKSSPGTSNKRLRQVSSLVLHIEEAHKLPVKHFTNPYCNIYLNSVQVAKTHAREGQNPVWSEEFVFDDLPPDINRFEITLSNKTKKSKDPDILFMRCQLSRLQKGHATDEWFLLSSHIPLKGIEPGSLRVRARYSMEKIMPEEEYSEFKELILQKELHVVYALSHVCGQDRTLLASILLRIFLHEKLESLLLCTLNDREISMEDEATTLFRATTLASTLMEQYMKATATQFVHHALKDSILKIMESKQSCELSPSKLEKNEDVNTNLTHLLNILSELVEKIFMASEILPPTLRYIYGCLQKSVQHKWPTNTTMRTRVVSGFVFLRLICPAILNPRMFNIISDSPSPIAARTLILVAKSVQNLANLVEFGAKEPYMEGVNPFIKSNKHRMIMFLDELGNVPELPDTTEHSRTDLSRDLAALHEICVAHSDELRTLSNERGAQQHVLKKLLAITELLQQKQNQYTKTNDVR

SEQ ID NO: 3 - DNA, 20 bp, Artificial Sequence
  probe or primer
actgacagggggaggtactg

SEQ ID NO: 4 - DNA, 20 bp, Artificial Sequence
  probe or primer
ttcccaaattcctgaacagc

SEQ ID NO: 5 - DNA, 18 bp, Artificial Sequence
  probe or primer
gggagctgaaggggagac

SEQ ID NO: 6 - DNA, 20 bp, Artificial Sequence
  probe or primer
ctacgccagcagcagtacct

SEQ ID NO: 7 - DNA, 24 bp, Artificial Sequence
  probe or primer
aagtgtccatagaaattctgcact

SEQ ID NO: 8 - DNA, 25 bp, Artificial Sequence
  probe or primer
catttggcttcataataggaataaa

SEQ ID NO: 9 - DNA, 24 bp, Artificial Sequence
  probe or primer
ggaaaagagtatggaaattatgga

SEQ ID NO: 10 - DNA, 27 bp, Artificial Sequence
  probe or primer
gcaatagctaaaaccattattgtactg

SEQ ID NO: 11 - DNA, 22 bp, Artificial Sequence
  probe or primer
tgaatgatcccatggagtttct

SEQ ID NO: 12 - DNA, 23 bp, Artificial Sequence
  probe or primer
cgaagtctagctctttcaaatgc

SEQ ID NO: 13 - DNA, 22 bp, Artificial Sequence
  probe or primer
tctgattacggacaagatccaa

SEQ ID NO: 14 - DNA, 20 bp, Artificial Sequence
  probe or primer
gtgtggggatatgtttgcag

SEQ ID NO: 15 - DNA, 25 bp, Artificial Sequence
  probe or primer
aaaagttaagtcagtccaaaacctt

SEQ ID NO: 16 - DNA, 27 bp, Artificial Sequence
  probe or primer
cactttgaattaaacttactatattgg

SEQ ID NO: 17 - DNA, 21 bp, Artificial Sequence
  probe or primer
tgctaaaggcaaacacatgat

SEQ ID NO: 18 - DNA, 25 bp, Artificial Sequence
  probe or primer
tgtttatgactttgaatgcactttg

SEQ ID NO: 19 - DNA, 24 bp, Artificial Sequence
  probe or primer
tttttgcgaaaagtaaaagatagc

SEQ ID NO: 20 - DNA, 22 bp, Artificial Sequence
  probe or primer
ccttggcaagaaagtttacaca

SEQ ID NO: 21 - DNA, 20 bp, Artificial Sequence
  probe or primer
tgtgcaaaaacataccacca

SEQ ID NO: 22 - DNA, 20 bp, Artificial Sequence
  probe or primer
agcgctttggcttttaattg

SEQ ID NO: 23 - DNA, 21 bp, Artificial Sequence
  probe or primer
ttcggagctccatatttacaa

SEQ ID NO: 24 - DNA, 23 bp, Artificial Sequence
  probe or primer
gctttggaataaaaattgattga

SEQ ID NO: 25 - DNA, 23 bp, Artificial Sequence
  probe or primer
ttccgaaagaaaaataggaaacc

SEQ ID NO: 26 - DNA, 20 bp, Artificial Sequence
  probe or primer
tgagtgttttggaagctggt

SEQ ID NO: 27 - DNA, 21 bp, Artificial Sequence
  probe or primer
tttcaggcgttctgtcacttt

SEQ ID NO: 28 - DNA, 23 bp, Artificial Sequence
  probe or primer
gaaatggcagtctagagaaggaa

SEQ ID NO: 29 - DNA, 23 bp, Artificial Sequence
  probe or primer
gcaaagtgttagagcaaaatgtg

SEQ ID NO: 30 - DNA, 27 bp, Artificial Sequence
  probe or primer
ttttggctttgtatcttagagtaattg

SEQ ID NO: 31 - DNA, 22 bp, Artificial Sequence
  probe or primer
acagaaagaaatgcaatatggt

SEQ ID NO: 32 - DNA, 22 bp, Artificial Sequence
  probe or primer
gcagaaatagggggtttatttg

SEQ ID NO: 33 - DNA, 24 bp, Artificial Sequence
  probe or primer
aagactttcattgtgaattttgaa

SEQ ID NO: 34 - DNA, 20 bp, Artificial Sequence
  probe or primer
gggaagactgaacaccagga

SEQ ID NO: 35 - DNA, 22 bp, Artificial Sequence
  probe or primer
ttccaacaaaaacaagactgat

SEQ ID NO: 36 - DNA, 23 bp, Artificial Sequence
  probe or primer
tttcttgttagtctcatggagca

SEQ ID NO: 37 - DNA, 25 bp, Artificial Sequence
  probe or primer
aaacccagtttcttgtatcacacta

SEQ ID NO: 38 - DNA, 22 bp, Artificial Sequence
  probe or primer
ccaatttggtcacattaggtca

SEQ ID NO: 39 - DNA, 25 bp, Artificial Sequence
  probe or primer
ttttccttaaaatgtaattggctac

SEQ ID NO: 40 - DNA, 25 bp, Artificial Sequence
  probe or primer
caacctcgaaaactataactacttg

SEQ ID NO: 41 - DNA, 23 bp, Artificial Sequence
  probe or primer
aacagaaaagctttcacgtttta

SEQ ID NO: 42 - DNA, 20 bp, Artificial Sequence
  probe or primer
tggctgctaggagatcagtg

SEQ ID NO: 43 - DNA, 20 bp, Artificial Sequence
  probe or primer
tgcaacagggctttgacata

SEQ ID NO: 44 - DNA, 25 bp, Artificial Sequence
  probe or primer
tgggttctatgagtactaaaaattc

SEQ ID NO: 45 - DNA, 24 bp, Artificial Sequence
  probe or primer
tgactagaattggatgatcaaaaa

SEQ ID NO: 46 - DNA, 21 bp, Artificial Sequence
  probe or primer
ggtttagctggaagtgctgtt

SEQ ID NO: 47 - DNA, 23 bp, Artificial Sequence
  probe or primer
tggttttatcatgtcaaacttgc

SEQ ID NO: 48 - DNA, 24 bp, Artificial Sequence
  probe or primer
tttgcaccaacctaatagatcaaa

SEQ ID NO: 49 - DNA, 21 bp, Artificial Sequence
  probe or primer
gattgctgcttaaatgggtta

SEQ ID NO: 50 - DNA, 20 bp, Artificial Sequence
  probe or primer
ggcaacaagagcgaaactct

SEQ ID NO: 51 - DNA, 25 bp, Artificial Sequence
  probe or primer
aagtgttatctttgaaacatcattg

SEQ ID NO: 52 - DNA, 20 bp, Artificial Sequence
  probe or primer
ttcaaatccaggttcccatc

SEQ ID NO: 53 - DNA, 21 bp, Artificial Sequence
  probe or primer
gctgaatccatgcagaacact

SEQ ID NO: 54 - DNA, 20 bp, Artificial Sequence
  probe or primer
ggacgaaggtgactctctgg

SEQ ID NO: 55 - DNA, 22 bp, Artificial Sequence
  probe or primer
caaaccacagatgaaaaggaca

SEQ ID NO: 56 - DNA, 24 bp, Artificial Sequence
  probe or primer
gtttttattttaaaggccaaacta

SEQ ID NO: 57 - DNA, 24 bp, Artificial Sequence
  probe or primer
gtttttattttaaaggccaaactg

SEQ ID NO: 58 - DNA, 22 bp, Artificial Sequence
  probe or primer
tgctgaactactatctgaaaat

SEQ ID NO: 59 - DNA, 22 bp, Artificial Sequence
  probe or primer
tgctgaactactatctgaaaac

SEQ ID NO: 60 - DNA, 22 bp, Artificial Sequence
  probe or primer
cagcttcaatctgtttgtaact